(19) United States
(12) Patent Application Publication
(10) Pub. No.: US 2021/0293833 A1
(43) Pub. Date: Sep. 23, 2021

(54) METHOD FOR SCREENING ADULTERATION OF FIBRATE ANTI-HYPERLIPIDEMIA CHEMICALS IN TEA BY COMBINED METHOD OF HIGH PERFORMANCE THIN LAYER CHROMATOGRAPHY AND BIOLUMINESCENCE

(71) Applicant: Jiangnan University, Wuxi (CN)

(72) Inventors: Yisheng CHEN, Wuxi (CN); Caihong HUANG, Wuxi (CN); Lanping SHU, Wuxi (CN); Xueming XU, Wuxi (CN); Zhengyu JIN, Wuxi (CN); Jie LONG, Wuxi (CN); Yaoqi TIAN, Wuxi (CN); Yuxiang BAI, Wuxi (CN); Na YANG, Wuxi (CN); Yamei JIN, Wuxi (CN)

(73) Assignee: Jiangnan University, Wuxi (CN)

(21) Appl. No.: 16/964,593

(22) PCT Filed: May 24, 2019
(86) PCT No.: PCT/CN2019/088357

§ 371 Date: Jul. 23, 2020

(30) Foreign Application Priority Data
Apr. 11, 2019 (CN) 201910287919.6

(51) Int. Cl. G01N 33/92; G01N 30/95

(52) U.S. Cl. CPC G01N 33/92; G01N 2570/00; G01N 30/95

(57) ABSTRACT

A method for screening adulteration of fibrate anti-hyperlipidemia chemicals in tea by combined method of high performance thin layer chromatography (HPTLC) and bioluminescence, which belongs to the field of food inspection. The method includes: firstly, formulating standard solutions of bezafibrate and ciprofibrate, and preparing a tea sample; pre-washing a thin-layer plate and then performing HPTLC spotting; performing HPTLC separation to move original mixed target objects mixed originally onto different positions of the thin-layer plate according to different molecular structures, so as to form a physical isolation; and subsequently, simultaneous detection of multiple targets in the tea sample could be realized conveniently through luminous bacteria coupled with the thin-layer plate by an immersed manner. The present disclosure establishes a method capable of detecting anti-hyperlipidemia chemicals in tea rapidly and quantitatively by the combined detection method of HPTLC and bioluminescence, which has the advantages of being economic, rapid, simple and convenient.

### BACKGROUND OF THE INVENTION

### Field of the Invention

The present disclosure, belonging to the field of food inspection, relates to a method for screening adulteration of fibrate anti-hyperlipidemia chemicals in tea by combined method of high performance thin layer chromatography (HPTLC) and bioluminescence, and in particular, relates to a combined method of high-throughput screening and bioluminescence inhibition imaging, which applies high-throughput screening of adulteration of illegal chemicals added in different tea products.

### Description of the Related Art

“Affluence Diseases” caused by unscientific daily diet has become the most important threat to human health instead of pathogenic microorganism infection. Hyperlipidemia is one of the most common “affluence disease”, which not only has a serious negative impact on human health directly, but also easily leads to complications such as a coronary heart disease, hypertension, cerebral thrombosis, atherosclerosis, etc., and has become a “hidden killer” of human health in the modern society.

Tea is one of the three traditional beverages in the world. In addition to its widely accepted taste, the health effects of tea have been paid more and more attention by researchers of food science and technology. More and more studies have proved that tea is rich in bio-active substances such as polysaccharides, flavonoids and polyphenols, which has good effects on regulating blood lipid metabolism and relieving hyperlipidemia.

Under this background, the market space of health-care tea products with good anti-hyperlipidemia effect is experiencing rapid expanding. In particular, some types of health-care tea derived from raw materials that can be used as both food and medicine have even become “online celebrity products”, but the accompanying food and medicine safety problems have also occurred in a blowout manner. According to the cases disclosed by the media and the food and drug administration departments, some lawbreakers have illegally added cheap chemicals with similar efficacy into tea in order to achieve the anti-hyperlipidemia effect claimed in the advertisements of the product. Bezafibrate (BZF) and ciprofibrate (CPF) belong to fibrate anti-hyperlipidemia chemicals, whose principle of pharmaceutical effect of them is accelerating the decomposition of lipoproteins by enhancing the activity of a lipoprotein lipase, and meanwhile reducing the synthesis of lipoproteins in the liver, so as to achieve the anti-hyperlipidemia effect. The fibrate anti-hyperlipidemia chemicals have good efficacy and less side effects, which can be easily used for adulteration in the health-care tea. These behaviors are not only suspected of commercial fraud, but also cause serious poisoning and side effects, and even endanger life since the chemicals are added in a large dosage and arbitrarily and consumers often take them for a long time because of the temporary significant effect without knowing the adulteration. At present, there is no special detection item of adulteration of illegal chemicals in the tea in China, therefore, it is of great significance to establish a method for screening and detecting adulteration of anti-hyperlipidemia chemicals in tea.

Luminous bacteria is a class of microorganisms capable of emitting visible light under normal physiological conditions. The luminescence principle of such bacteria is that under the catalysis of a luciferase and the action of molecular oxygen, long-chain fatty aldehydes and reduced flavin mononucleotide (FMNH₂) are oxidized to long-chain fatty acids and oxidized flavin mononucleotide (FMN), and emitting blue-green light with a wavelength of 450-490 nm simultaneously. Because of this characteristic, the luminous bacteria is often used as optical sensing elements of biodetectors. Compared with the common physical and chemical detection means, the biggest advantage of biosensing by the luminous bacteria is the non-targeting detection ability: its sensing principle is to characterize the toxicity of target objects by the degrees of bioluminescence inhibition, rather than based on the identification of specific chemical structure. More specifically, when there is an interference of toxic substances in the external environment, the physiological process or cell respiration of the luminous bacteria is affected, which leads to the inhibition of the luminescence reaction. There is a correlation between the content of the toxic substances and the weakening degree of the luminous intensity of the bacteria.

Compared with the toxicity test methods of model organisms such as zebrafishes, nematodes and the like, which are complicated in preparation work and have long period, the advantages of the luminous bacteria method are simple in operation (the freeze-dried bacteria powder can be used directly after resurrection), rapid and efficient (the results can be obtained within several minutes at the soonest), so the luminous bacteria method has a broader applied prospect. At present, the analysis method based on the luminous bacteria has played an important role in the safety and early-warning system for drinking water of European Union. In China, the detecting technology of luminous bacteria played an important role in the work of emergency insurance of water quality in the “5.12 Wenchuan Earthquake” disaster areas, monitoring the water quality of Taihu Lake, and the like.

Generally, a photodensitometer or a microplate reader with a luminescence analysis function is an instrument carrier for the application of the luminous bacteria method. Analysts only need to mix the bacteria solution with the sample, and quantitatively detect the change of luminous intensity after a period of reaction. Although this mode is simple and efficient, it is not suitable for the detection of illegal addition of chemical medicines in the tea because of the following two serious defects: serious background interference and lack of selectivity for multiple targets.

HPTLC is the only chromatographic tool that can be used directly in combination with a cell-based biosensor. Different from a traditional tube testing method, the use in combination with HPTLC can fundamentally solve the problems of strong background interference and poor selectivity of the luminous bacteria method. HPTLC separation enables the original mixed target objects to move onto different positions of the thin-layer plates according to different molecular structures, so as to form physical isolation; and subsequently, simultaneous detection of multiple targets in the tea sample could be realized conveniently through luminous bacteria coupled with the thin-layer plate by an immersed manner. Therefore, the analytical method based on the combination of the two methods has the advantages of high selectivity and good generality, and has become an emerging hotspot and frontier in analytical chemistry, which playing an important role in many fields such as environmental monitoring and natural product analysis.

### BRIEF SUMMARY OF THE INVENTION

In view of the defects existing in the above prior arts, the present disclosure aims at providing a method for screening adulteration of fibrate anti-hyperlipidemia chemicals in tea by the combined method of high performance thin layer chromatography and bioluminescence.

The present disclosure adopts the following technical scheme. A method for screening adulteration of fibrate anti-hyperlipidemia chemicals in tea by the combined method of high performance thin layer chromatography and bioluminescence, including the steps of: formulating standard solutions of bezafibrate and ciprofibrate, preparing a tea sample; pre-washing thin-layer plate and then performing HPTLC spotting; performing HPTLC separation to move original mixed target objects onto different positions of the thin-layer plate according to different molecular structures, so as to form a physical isolation; and subsequently, simultaneous detection of multiple targets in the tea sample could be realized conveniently through luminous bacteria coupled with the thin-layer plate by an immersed manner.

The specific steps are as follows:

(1) formulation of standard solutions: precisely weighing 10 mg of bezafibrate standard and 10 mg of ciprofibrate standard with an electronic balance, placed into a 10 mL volumetric flask respectively, and adjusting to a constant volume with methanol to obtain a standard stock solution with a concentration of 1 mg/mL; at normal times, storing the standard stock solution standing at 4° C. in dark, before the analysis working beginning, taking 0.2 mL of the standard stock solution into a 10 mL volumetric flask, and diluted with methanol to a constant volume, so as to obtain a standard working solution with a concentration of 0.02 mg/mL; the standard working solution is ready-to-use;

(2) preparation of tea sample solutions respectively: firstly, taking Folium nelumbinis, Apocynum venetum and Ginkgo biloba, uniformly crushed with an electric crusher respectively; then weighing 1.0 g of the crushed sample, 10 mL of methanol is added, ultrasonic extracting in a water bath at 25° C. for 30 min, taken out and then centrifuged at 5000 r/min for 5 min; after the centrifugation, taking 5 mL of supernatant of the upper layer and charging it into a syringe, compressing plunger rod of the syringe to enable the supernatant to pass through a 0.45 μm nylon filter membrane, and the resulting sample supernatant could be directly used for HPTLC spotting;

(3) HPTLC spotting and chromatographic conditions: firstly, manually pipetting the sample solution with a 100 μL spotting needle; spotting by a semi-automatic thin-layer spotting device, with the assistance of a 0.5 MPa nitrogen flow, sweeping to a position 10 cm away from the bottom of the thin-layer plate with a liquid flow sweeping speed of 100 μL/s, a pre-discharge volume of 0.2 μL, a band width of 6 mm and a distance from both side edges of at least 15 mm; spotting each of the samples prepared by the steps (1) and (2), after finishing the spotting of one sample, manually taking out the spotting needle, washing it with methanol for three times, and then carrying out the spotting of a next sample; after finishing all the spotting, taking out the thin-layer plate and heated with an electric hair dryer for 1 min to volatilize the residual methanol attached at spotting positions;

chromatographic separation is carried out in a full-automatic thin layer chromatographic expander with a mobile phase ration: a volume ratio of ethyl acetate/methanol being 9/1, and an expanding distance being 60 mm; the chromatographic separation conditions: controlling relative humidity in an expanding tank by bubbling a saturated magnesium chloride solution for 3 min, adjust the relative humidity to 35%, and pre-equilibrating the thin-layer plate for 10 min; when a front edge of the mobile phase reaching a predetermined height, the system automatically ending, taking out the thin-layer plate and baking it on a thin-layer heater at 80° C. for 5 min to obtain a bright and low-noise bioluminescence imaging background;

(4) detection by bioluminescence imaging: immersing the expanded and dried thin-layer plate into a luminescent working suspension by using an automatic immersion device at an immersion speed of 1 mm/s and a retention time of 2 s, then putting the thin-layer plate immersed with the luminescent working suspension into a bioluminescence imager for detection by imaging, with a imaging exposure time of 40 s, a imaging interval of 2 min, and taking 15 photographs continuously;

(5) analysis: saving the photographs taken by the bioluminescence imager, opening the photographs with a Videoscan software, digitizing the pixel gray scale in the photographs, and then setting integral parameters and conditions for quantitative analysis.

Further, the preparation process of the luminescent working suspension is as follows:

(1) formulation of simulated seawater liquid medium:

formulating a simulated seawater liquid medium according to the following formula: 30 g/L of NaCl, 5 g/L of Na₂HPO₄, 5 g/L of KH₂PO₄, 3 mL/L of glycerol, 5 g/L of peptone and 5 g/L of a yeast extract, dissolving under stirring by adding 1 L of ultrapure water; adjusting the pH value of the formulated liquid medium to 7.3-7.7 with 1 mol/L of sodium hydroxide solution, and carrying out sterilization treatment at 121° C. for 15 min within a high-pressure steam sterilization pot to obtain a simulated seawater liquid medium, then packaging the simulated seawater liquid medium and refrigerating it in a refrigerator for later use, and the simulated seawater liquid medium could be stored in an environment of 4° C. for 7 days when being idle; and

(2) culture and preservation of luminous strains: inoculating luminous bacteria cryopreserved with glycerol into a triangular flask containing 100 mL of the liquid medium prepared in the step (1); wrapping the flask mouth with sterilized four-layer folded tin foil paper to ensure external oxygen could enter the flask during the culture process, and then culturing the bacteria in the flask under shaking at 100 r/min in an environment of 25° C. to obtain a bacterial mother liquor; then adding an equal volume of fresh liquid medium into the mature bacterial mother liquor to prepare a luminescent working suspension; and the luminescent working suspension can be stored in an environment of 4° C. for 3 days when being idle.

Further, the luminous strains according to step (2) are preserved by an agar plate method, specifically as follows:

a. preparation of luminous bacteria medium: taking 10 g of agar, 30 g of NaCl, 5 g of Na₂HPO₄, 5 g of KH₂PO₄, 3 mL of glycerol, 5 g of peptone and 5 g of a yeast extract, and adding 1 L of ultrapure water to dissolve under stirring, then adjusting the pH value to 7.5±0.2 with a 1 mol/L sodium hydroxide aqueous solution, and then carrying out sterilization treatment at 121° C. for 15 min within a high-pressure steam sterilization pot; and when the sterilized culture medium is cooled to 60° C., pouring it into a Petri dish with a diameter of 10 cm to form a plate while hot; and

b. inoculation: firstly, immersing a sterilized inoculating loop into a mature luminous bacteria medium, and then shading with diagonal lines on the surface of the nutrient agar medium, repeating it for many times; culturing the inoculated nutrient agar medium in dark in an environment of 25° C. for 48 h, a bacterial mother liquor is obtained after obvious colonies are observed, and then transferring it into an environment of −4° C. in dark for preservation.

Further, in step (2), the absorbance of the liquid medium to an incident light at 600 nm is taken as an index for determining the cell density; a clear simulated seawater liquid medium is taken as a reference, the absorbance OD₆₀₀ of the medium to the incident light at 600 nm is monitored by a spectrophotometer during the culture process, and the culture solution is selected as the bacterial mother liquor when the numerical value of OD₆₀₀ reaches 0.7.

Further, the thin-layer plate in step (3) needs to be pre-washed, specifically as follows: firstly, pouring 10 mL of methanol into a clean expanding tank, then a blank thin-layer plate being put; expanding the thin-layer plate to the top end thereof, keeping for 5 min when the thin-layer plate expanded to the top end to wash off impurities as many as possible; then taking out the thin-layer plate and baking it on a thin-layer plate heater at 100° C. for 5 min to volatilize the residual organic solvent; and wrapping the dried thin-layer plate with aluminum foil paper for later use.

Further, a thin-layer material of the thin-layer plate is an ordinary silica gel.

Further, the specific process of the step (5) is as follows: saving the photographs taken by the bioluminescence imager in CPF, Black/white linear formats, opening the photographs with a Videoscan software, digitizing the pixel gray scale in the photographs, and then setting integral parameters and conditions for quantitative analysis.

Further, the luminous bacteria, a class of microorganisms, could be emitting visible light under normal physiological conditions. The luminescence principle of such bacteria is that under the catalysis of a luciferase and the action of molecular oxygen, long-chain fatty aldehydes and reduced flavin mononucleotide (FMNH2) are oxidized into long-chain fatty acids and oxidized flavin mononucleotide (FMN), simultaneously, releasing blue-green light with a wavelength of 450-490 nm. Based on this characteristic, the luminous bacteria is often used as optical sensing elements of biodetectors. Compared with the common physical and chemical detection means, the biggest advantage of biosensing by the luminous bacteria lies in the non-targeting detection ability; its sensing principle is to characterize the toxicity of target objects by the degrees of bioluminescence inhibition, rather than based on the identification of a specific chemical structure.

The present disclosure has the following prominent technical effects: the present disclosure establishes a method capable of detecting anti-hyperlipidemia chemicals in tea rapidly and quantitatively by combined method of HPTLC and bioluminescent, and has the advantages of being economic, rapid, simple and convenient.

### BRIEF DESCRIPTION OF THE DRAWINGS

FIG. 1 is an imaging diagram of separation results under the conditions of bacterial liquor immersed-bioluminescence inhibition (pseudo-color mode) in Examples 1-3.

Spotting tracks: 1. BZF, 2. CPF, 3. Folium nelumbinis, 4. Folium nelumbinis+standard, 5. Ginkgo biloba, 6. Ginkgo biloba+standard.

FIG. 2 is a standard curve of BZF.

FIG. 3 is a standard curve of CPF.

Table 1 is the evaluation of the quantitative detection abilities of the methods.

Table 2 is the evaluation of the quantitative accuracy of the methods.

### DETAILED DESCRIPTION OF THE INVENTION

In the following examples, the analytical standards of ciprofibrate (≥99%, HPLC) and bezafibrate (≥96.0%, HPLC) are purchased from Aladdin (Shanghai, China). The analytically pure NaCl, Na₂SO₄, NaNO₃ and other chemical reagents are purchased from Sigma-Aldrich. The high-efficiency thin-layer silica gel plate with a glass substrate (analytical type, size 10×20 cm, batch number 1.05729.0001) is purchased from Merck (Darmstadt, Germany). The thin-layer plate is washed once with methanol before use. Blank tea samples are purchased from local supermarkets.

The luminous bacterium used in the following examples is Aliivibrio fischeri (strain preservation number DSM 507), and the strain is purchased from the China General Microbiological Culture Collection Center, abbreviated as CGMCC.

### Example 1

(1) formulation of simulated seawater liquid and solid medium: formulating a simulated seawater liquid medium according to the following formula: 30 g/L of NaCl, 5 g/L of Na₂HPO₄, 5 g/L of KH₂PO₄, 3 ml/L of glycerol, 5 g/L of peptone, and 5 g/L of a yeast extract, adding 1 L of ultrapure water to dissolve under stirring; adjusting the pH value to 7.5±0.2 with 1 mol/L of sodium hydroxide solution, and carrying out sterilization treatment at 121° C. for 15 min within a high-pressure steam sterilization pot to obtain a simulated seawater liquid medium, then packaging the simulated seawater liquid medium and refrigerating it in a refrigerator for later use, and the simulated seawater liquid medium could be stored in an environment of 4° C. for 7 days when being idle; and

(2) culture and preservation of luminous strains: inoculating luminous bacteria cryopreserved with glycerol into a triangular flask containing 100 mL of the liquid medium prepared in the step (1); wrapping the flask mouth with sterilized four-layer folded tin foil paper to ensure external oxygen could enter the flask during the culture process, and then culturing the bacteria in the flask under shaking at 100 r/min in an environment of 25° C. according to the method described by Chen et al. to obtain a bacterial mother liquor; then adding an equal volume of fresh liquid medium into the mature bacterial mother liquor to prepare a luminescent working suspension; and the luminescent working suspension can be stored in an environment of 4° C. for 3 days when being idle.

The luminous strains of bacteria were preserved by an agar plate method, and the method for preparing the nutrient agar plate was as follows: taking 10 g of agar, 30 g of NaCl, 5 g of Na₂HPO₄, 5 g of KH₂PO₄, 3 mL of glycerol, 5 g of peptone and 5 g of a yeast extract and adding 1 L of ultrapure water to dissolve under stirring, then adjusting the pH value to 7.5±0.2 with a 1 mol/L sodium hydroxide aqueous solution, and then carrying out sterilization treatment at 121° C. for 15 min within a high-pressure steam sterilization pot; and when a sterilized culture medium cooled to 60° C., pouring it into a Petri dish with a diameter of 10 cm to form a plate while hot; and inoculation: firstly, immersing a sterilized inoculating loop into a mature luminous bacteria medium, and then shading with diagonal lines on the surface of the nutrient agar medium, repeating it for many times; culturing the inoculated nutrient agar medium in dark in an environment of 25° C. for 48 h, obtaining a bacterial mother liquor when obvious colonies being observed, and then transferring it into an environment of −4° C. in dark for preservation.

(3) Formulation of standard solutions: precisely weighing 10±0.1 mg of bezafibrate standard and 10±0.1 mg of ciprofibrate standard with an electronic balance, respectively placed into a 10 mL volumetric flask, and adjusting to a constant volume with methanol to obtain a standard stock solution with a concentration of 1 mg/mL; at normal times, storing the standard stock solution standing at 4° C. in dark, immediately before the analysis working time, taking 0.2 mL of the standard stock solution into a 10 mL volumetric flask, and diluted with methanol to a constant volume, so as to obtain a standard working solution with a concentration of 0.02 mg/mL; the standard working solution is ready-to-use;

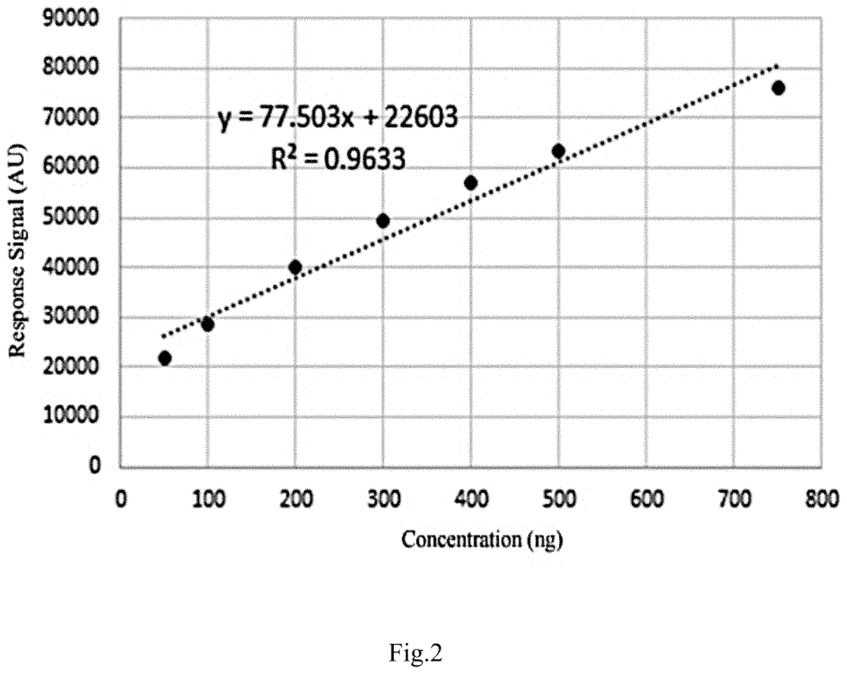

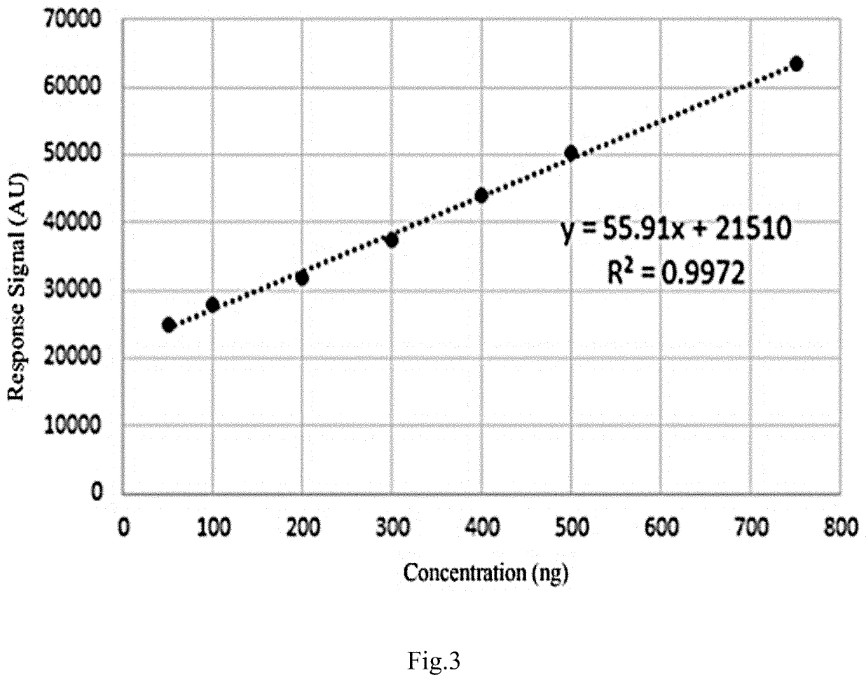

The standard curve of BZF was shown in FIG. 2, and the standard curve of CPF was shown in FIG. 3.

(4) Pre-washing of thin-layer plate: the small amount of organic residues in the stationary phase of the thin-layer plate would have a significant negative impact on the bioluminescence of the bacteria, so it was necessary to pre-wash the thin-layer plate with methanol before use. Firstly, pouring 10 mL of methanol into a clean expanding tank, then a blank thin-layer plate being put; expanding the thin-layer plate to the top end thereof, keeping for 5 min when the thin-layer plate expanded to the top end to wash off impurities as many as possible; then taking out the thin-layer plate and baking it on a thin-layer plate heater at 100° C. for 5 min to volatilize the residual organic solvent; and wrapping the dried thin-layer plate with aluminum foil paper for later use;

(5) HPTLC spotting and chromatographic conditions: firstly, manually pipetting the sample solution with a 100 μL spotting needle; spotting by a semi-automatic thin-layer spotting device, with the assistance of a 0.5 MPa nitrogen flow, sweeping the sample solution to a position 10 cm away from the bottom of the thin-layer plate with a liquid flow sweeping speed of 100 μL/s, a pre-discharge volume of 0.2 μL, a band width of 6 mm and a distance from both side edges of at least 15 mm; spotting each of the samples prepared by the step (3), after finishing the spotting of one sample, manually taking out the spotting needle, washing it with methanol for three times, and then carrying out the spotting of a next sample; after finishing all the spotting, taking out the thin-layer plate and heated with an electric hair dryer for 1 min to volatilize the residual methanol attached at spotting positions.

Chromatographic separation was carried out in a full-automatic thin layer chromatographic expander with a mobile phase ration: a volume ratio of ethyl acetate/methanol being 9/1 and an expanding distance being 60 mm. The chromatographic separation conditions: the relative humidity in an expanding tank was controlled by bubbling a saturated magnesium chloride solution for 3 min (the RH was adjusted to about 35% by bubbling the saturated magnesium chloride solution), the thin-layer plate was pre-equilibrated for 10 min, and it took about 25 min for the whole expanding process. When a front edge of the mobile phase reached a predetermined height, the system automatically ended, and the thin-layer plate was taken out and baked on a thin-layer heater at 80° C. for 5 min to volatilize the residual organic solvent to obtain a bright and low-noise bioluminescence imaging background.

(6) Detection by bioluminescence imaging: immersing the expanded and dried thin-layer plate into a luminescent working suspension by using an automatic immersion device at an immersion speed of 1 mm/s for a retention time of 2 s, and then putting the thin-layer plate immersed with the luminescent working suspension into a bioluminescence imager to imaging detect, with a imaging exposure time of 40 s, a imaging interval of 2 min, and 15 photographs taken continuously; and

(7) saving the photographs taken by the bioluminescence imager in CPF (Black/white linear) format, opening the photographs with a Videoscan software, digitizing the pixel gray scale in the photographs, and then setting integral parameters and conditions for quantitative analysis.

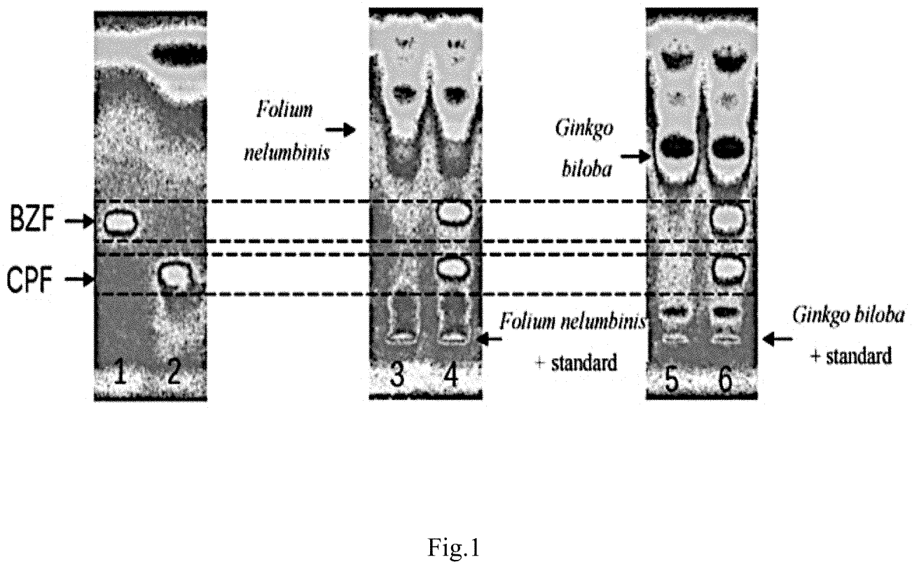

The imaging diagram of separation results under the conditions of bacterial liquor immersed-bioluminescence inhibition (pseudo-color mode) was shown in FIG. 1.

### Example 2

(1) formulation of simulated seawater liquid and solid medium: formulating a simulated seawater liquid medium according to the following formula: 30 g/L of NaCl, 5 g/L of Na₂HPO₄, 5 g/L of KH₂PO₄, 3 ml/L of glycerol, 5 g/L of peptone, and 5 g/L of a yeast extract, adding 1 L of ultrapure water to dissolve under stirring; adjusting the pH value to 7.5±0.2 with 1 mol/L of sodium hydroxide solution, and carrying out sterilization treatment at 121° C. for 15 min within a high-pressure steam sterilization pot to obtain a simulated seawater liquid medium, then packaging the simulated seawater liquid medium and refrigerating it in a refrigerator for later use, and the simulated seawater liquid medium could be stored in an environment of 4° C. for 7 days when being idle; and

(2) culture and preservation of luminous strains: inoculating luminous bacteria cryopreserved with glycerol into a triangular flask containing 100 mL of the liquid medium prepared in the step (1); wrapping the flask mouth with sterilized four-layer folded tin foil paper to ensure external oxygen could enter the flask during the culture process, and then culturing the bacteria in the flask under shaking at 100 r/min in an environment of 25° C. according to the method described by Chen et al. to obtain a bacterial mother liquor; then adding an equal volume of fresh liquid medium into the mature bacterial mother liquor to prepare a luminescent working suspension; and the luminescent working suspension can be stored in an environment of 4° C. for 3 days when being idle.

The luminous strains of bacteria were preserved by an agar plate method, and the method for preparing the nutrient agar plate was as follows: taking 10 g of agar, 30 g of NaCl, 5 g of Na₂HPO₄, 5 g of KH₂PO₄, 3 mL of glycerol, 5 g of peptone and 5 g of a yeast extract and adding 1 L of ultrapure water to dissolve under stirring, then adjusting the pH value to 7.5±0.2 with a 1 mol/L sodium hydroxide aqueous solution, and then carrying out sterilization treatment at 121° C. for 15 min within a high-pressure steam sterilization pot; and when a sterilized culture medium cooled to 60° C., pouring it into a Petri dish with a diameter of 10 cm to form a plate while hot; and inoculation: firstly, immersing a sterilized inoculating loop into a mature luminous bacteria medium, and then shading with diagonal lines on the surface of the nutrient agar medium, repeating it for many times; culturing the inoculated nutrient agar medium in dark in an environment of 25° C. for 48 h, obtaining a bacterial mother liquor when obvious colonies being observed, and then transferring it into an environment of −4° C. in dark for preservation.

(3) Formulation of standard solutions: precisely weighing 10±0.1 mg of bezafibrate standard and 10±0.1 mg of ciprofibrate standard with an electronic balance, respectively placed into a 10 mL volumetric flask, and adjusting to a constant volume with methanol to obtain a standard stock solution with a concentration of 1 mg/mL; at normal times, storing the standard stock solution standing at 4° C. in dark, immediately before the analysis working time, taking 0.2 mL of the standard stock solution into a 10 mL volumetric flask, and diluted with methanol to a constant volume, so as to obtain a standard working solution with a concentration of 0.02 mg/mL; the standard working solution is ready-to-use;

The standard curve of BZF was shown in FIG. 2, and the standard curve of CPF was shown in FIG. 3.

(4) Preparation of a sample: firstly, taking and uniformly crushing a sample of Folium nelumbinis with an electric crusher; then weighing 1.0 g of the crushed sample and added it into 10 mL of methanol, ultrasonic extracting in a water bath at 25° C. for 30 min, taken out and then centrifuged at 5000 r/min for 5 min; after the centrifugation, taking 5 mL of supernatant of the upper layer and charging it into a syringe, compressing a plunger rod of the syringe to enable the supernatant to pass through a 0.45 μm nylon filter membrane, and the resulting sample supernatant could be directly used for HPTLC spotting;

(5) pre-washing of thin-layer plate: the small amount of organic residues in the stationary phase of the thin-layer plate would have a significant negative impact on the bioluminescence of the bacteria, so it was necessary to pre-wash the thin-layer plate with methanol before use. Firstly, pouring 10 mL of methanol into a clean expanding tank, then a blank thin-layer plate being put; expanding the thin-layer plate to the top end thereof, keeping for 5 min when the thin-layer plate expanded to the top end to wash off impurities as many as possible; then taking out the thin-layer plate and baking it on a thin-layer plate heater at 100° C. for 5 min to volatilize the residual organic solvent; and wrapping the dried thin-layer plate with aluminum foil paper for later use;

(6) HPTLC spotting and chromatographic conditions: firstly, manually pipetting the sample solution with a 100 μL spotting needle; spotting by a semi-automatic thin-layer spotting device, with the assistance of a 0.5 MPa nitrogen flow, sweeping the sample solution to a position 10 cm away from the bottom of the thin-layer plate with a liquid flow sweeping speed of 100 μL/s, a pre-discharge volume of 0.2 μL, a band width of 6 mm and a distance from both side edges of at least 15 mm; spotting each of the samples prepared by the step (3), after finishing the spotting of one sample, manually taking out the spotting needle, washing it with methanol for three times, and then carrying out the spotting of a next sample; after finishing all the spotting, taking out the thin-layer plate and heated with an electric hair dryer for 1 min to volatilize the residual methanol attached at spotting positions.

Chromatographic separation was carried out in a full-automatic thin layer chromatographic expander with a mobile phase ration: a volume ratio of ethyl acetate/methanol being 9/1 and an expanding distance being 60 mm. The chromatographic separation conditions: the relative humidity in an expanding tank was controlled by bubbling a saturated magnesium chloride solution for 3 min, adjusting the RH to about 35%, the thin-layer plate was pre-equilibrated for 10 min, and it took about 25 min for the whole expanding process. When a front edge of the mobile phase reached a predetermined height, the system automatically ended, and the thin-layer plate was taken out and baked on a thin-layer heater at 80° C. for 5 min to volatilize the residual organic solvent to obtain a bright and low-noise bioluminescence imaging background.

(7) Detection by bioluminescence imaging: immersing the expanded and dried thin-layer plate into a luminescent working suspension by using an automatic immersion device at an immersion speed of 1 mm/s for a retention time of 2 s, and then putting the thin-layer plate immersed with the luminescent working suspension into a bioluminescence imager to imaging detect, with a imaging exposure time of 40 s, a imaging interval of 2 min, and 15 photographs taken continuously; and

(8) saving the photographs taken by the bioluminescence imager in CPF (Black/white linear) format, opening the photographs with a Videoscan software, digitizing the pixel gray scale in the photographs, and then setting integral parameters and conditions for quantitative analysis.

The imaging diagram of separation results under the conditions of bacterial liquor immersed-bioluminescence inhibition (pseudo-color mode) was shown in FIG. 1.

### Example 3

(1) formulation of simulated seawater liquid and solid medium: formulating a simulated seawater liquid medium according to the following formula: 30 g/L of NaCl, 5 g/L of Na₂HPO₄, 5 g/L of KH₂PO₄, 3 ml/L of glycerol, 5 g/L of peptone, and 5 g/L of a yeast extract, adding 1 L of ultrapure water to dissolve under stirring; adjusting the pH value to 7.5±0.2 with 1 mol/L of sodium hydroxide solution, and carrying out sterilization treatment at 121° C. for 15 min within a high-pressure steam sterilization pot to obtain a simulated seawater liquid medium, then packaging the simulated seawater liquid medium and refrigerating it in a refrigerator for later use, and the simulated seawater liquid medium could be stored in an environment of 4° C. for 7 days when being idle; and

(2) culture and preservation of luminous strains: inoculating luminous bacteria cryopreserved with glycerol into a triangular flask containing 100 mL of the liquid medium prepared in the step (1); wrapping the flask mouth with sterilized four-layer folded tin foil paper to ensure external oxygen could enter the flask during the culture process, and then culturing the bacteria in the flask under shaking at 100 r/min in an environment of 25° C. according to the method described by Chen et al. to obtain a bacterial mother liquor; then adding an equal volume of fresh liquid medium into the mature bacterial mother liquor to prepare a luminescent working suspension; and the luminescent working suspension can be stored in an environment of 4° C. for 3 days when being idle.

The luminous strains of bacteria were preserved by an agar plate method, and the method for preparing the nutrient agar plate was as follows: taking 10 g of agar, 30 g of NaCl, 5 g of Na₂HPO₄, 5 g of KH₂PO₄, 3 mL of glycerol, 5 g of peptone and 5 g of a yeast extract and adding 1 L of ultrapure water to dissolve under stirring, then adjusting the pH value to 7.5±0.2 with a 1 mol/L sodium hydroxide aqueous solution, and then carrying out sterilization treatment at 121° C. for 15 min within a high-pressure steam sterilization pot; and when a sterilized culture medium cooled to 60° C., pouring it into a Petri dish with a diameter of 10 cm to form a plate while hot; and inoculation: firstly, immersing a sterilized inoculating loop into a mature luminous bacteria medium, and then shading with diagonal lines on the surface of the nutrient agar medium, repeating it for many times; culturing the inoculated nutrient agar medium in dark in an environment of 25° C. for 48 h, obtaining a bacterial mother liquor when obvious colonies being observed, and then transferring it into an environment of −4° C. in dark for preservation.

(3) Formulation of standard solutions: precisely weighing 10±0.1 mg of bezafibrate standard and 10±0.1 mg of ciprofibrate standard with an electronic balance, respectively placed into a 10 mL volumetric flask, and adjusting to a constant volume with methanol to obtain a standard stock solution with a concentration of 1 mg/mL; at normal times, storing the standard stock solution standing at 4° C. in dark, immediately before the analysis working time, taking 0.2 mL of the standard stock solution into a 10 mL volumetric flask, and diluted with methanol to a constant volume, so as to obtain a standard working solution with a concentration of 0.02 mg/mL; the standard working solution is ready-to-use;

The standard curve of BZF was shown in FIG. 2, and the standard curve of CPF was shown in FIG. 3.

(4) Preparation of a sample: firstly, taking and uniformly crushing a sample of Ginkgo biloba with an electric crusher; then weighing 1.0 g of the crushed sample and added it into 10 mL of methanol, ultrasonic extracting in a water bath at 25° C. for 30 min, taken out and then centrifuged at 5000 r/min for 5 min; after the centrifugation, taking 5 mL of supernatant of the upper layer and charging it into a syringe, compressing a plunger rod of the syringe to enable the supernatant to pass through a 0.45 μm nylon filter membrane, and the resulting sample supernatant could be directly used for HPTLC spotting;

(5) pre-washing of thin-layer plate: the small amount of organic residues in the stationary phase of the thin-layer plate would have a significant negative impact on the bioluminescence of the bacteria, so it was necessary to pre-wash the thin-layer plate with methanol before use. Firstly, pouring 10 mL of methanol into a clean expanding tank, then a blank thin-layer plate being put; expanding the thin-layer plate to the top end thereof, keeping for 5 min when the thin-layer plate expanded to the top end to wash off impurities as many as possible; then taking out the thin-layer plate and baking it on a thin-layer plate heater at 100° C. for 5 min to volatilize the residual organic solvent; and wrapping the dried thin-layer plate with aluminum foil paper for later use;

(6) HPTLC spotting and chromatographic conditions: firstly, manually pipetting the sample solution with a 100 μL spotting needle; spotting by a semi-automatic thin-layer spotting device, with the assistance of a 0.5 MPa nitrogen flow, sweeping the sample solution to a position 10 cm away from the bottom of the thin-layer plate with a liquid flow sweeping speed of 100 μL/s, a pre-discharge volume of 0.2 μL, a band width of 6 mm and a distance from both side edges of at least 15 mm; spotting each of the samples prepared by the steps (3) and (4), after finishing the spotting of one sample, manually taking out the spotting needle, washing it with methanol for three times, and then carrying out the spotting of a next sample; after finishing all the spotting, taking out the thin-layer plate and heated with an electric hair dryer for 1 min to volatilize the residual methanol attached at spotting positions.

Chromatographic separation was carried out in a full-automatic thin layer chromatographic expander with a mobile phase ration: a volume ratio of ethyl acetate/methanol being 9/1 and an expanding distance being 60 mm. The chromatographic separation conditions: the relative humidity in an expanding tank was controlled by bubbling a saturated magnesium chloride solution for 3 min, (the RH was adjusted to about 35% by bubbling the saturated magnesium chloride solution), the thin-layer plate was pre-equilibrated for 10 min, and it took about 25 min for the whole expanding process. When a front edge of the mobile phase reached a predetermined height, the system automatically ended, and the thin-layer plate was taken out and baked on a thin-layer heater at 80° C. for 5 min to volatilize the residual organic solvent to obtain a bright and low-noise bioluminescence imaging background.

(7) Detection by bioluminescence imaging: immersing the expanded and dried thin-layer plate into a luminescent working suspension by using an automatic immersion device at an immersion speed of 1 mm/s for a retention time of 2 s, and then putting the thin-layer plate immersed with the luminescent working suspension into a bioluminescence imager to imaging detect, with a imaging exposure time of 40 s, a imaging interval of 2 min, and 15 photographs taken continuously; and

(8) saving the photographs taken by the bioluminescence imager in CPF (Black/white linear) format, opening the photographs with a Videoscan software, digitizing the pixel gray scale in the photographs, and then setting integral parameters and conditions for quantitative analysis.

The imaging diagram of separation results under the conditions of bacterial liquor immersed-bioluminescence inhibition (pseudo-color mode) was shown in FIG. 1.

### Example 4

Apocynum venetum was selected as the sample in the step (4), and other steps were the same as those in Example 3.

### Example 5

The detection abilities of the methods described in Examples 1-4 were evaluated. The specific results of quantitative detection ability evaluation were shown in Table 1, and the quantitative accuracy evaluation were shown in Table 2.

TABLE 1
Evaluation of quantitative detection abilities of methods

| | | | Linear Quantifying | | Binomial Quantifying | |
|---|---|---|---|---|---|---|
| | Quantitative Range | | Regression | | Regression | |
| Analyte | [ng/zone] | [mg/g] | Equation | R² | Equation | R² |
| BZF | 50-1000 | 0.05-1 | y = 62.789x + | 0.9279 | y = −0.0621x2 + | 0.9992 |
| | | | 26312 | | 126.59x + 16409 | |
| CPF | 50-1000 | 0.05-1 | y = 62.209x + | 0.9837 | y = 0.0242x2 + | 0.9955 |
| | | | 20305 | | 37.32x + 24168 | |

TABLE 2
Evaluation of the quantitative accuracy of methods

| | | | Recovery rate % | | |
|---|---|---|---|---|---|
| | Additive amount | | Folium | Apocynum | Ginkgo |
| Analyte | [ng/zone] | [μg/g] | nelumbinis | venetum | biloba |
| BZF | 50 | 5 | 81 | 79 | 86 |
| | 100 | 10 | 90 | 89 | 85 |
| | 200 | 20 | 82 | 84 | 88 |
| CPF | 50 | 5 | 74 | 78 | 83 |
| | 100 | 10 | 89 | 90 | 83 |
| | 200 | 20 | 78 | 83 | 91 |

What is claimed is:

1. A method for screening adulteration of fibrate anti-hyperlipidemia chemicals in tea by combined method of high performance thin layer chromatography and bioluminescence, comprising the steps of:
formulating standard solutions of bezafibrate and ciprofibrate, preparing a tea sample;
pre-washing a thin-layer plate and then performing HPTLC spotting;
performing HPTLC separation to move original mixed target objects onto different positions of the thin-layer plate according to different molecular structures, so as to form a physical isolation; and
subsequently, simultaneous detection of multiple targets in the tea sample could be realized conveniently through luminous bacteria coupled with the thin-layer plate by an immersed manner.

2. The method for screening adulteration of fibrate anti-hyperlipidemia chemicals in tea by combined method of high performance thin layer chromatography and bioluminescence according to claim 1, wherein the specific steps are as follows:
(1) formulation of standard solutions:
precisely weighing 10 mg of bezafibrate standard and 10 mg of ciprofibrate standard with an electronic balance, placed into a 10 mL volumetric flask respectively, and adjusting to a constant volume with methanol to obtain a standard stock solution with a concentration of 1 mg/mL;
at normal times, storing the standard stock solution standing at 4° C. in dark, before the analysis working begin, taking 0.2 mL of the standard stock solution into a 10 mL volumetric flask, and diluted with methanol to a constant volume, so as to obtain a standard working solution with a concentration of 0.02 mg/mL;
the standard working solution is ready-to-use;
(2) preparation of tea sample solutions respectively:
firstly, taking Folium nelumbinis, Apocynum venetum and Ginkgo biloba, uniformly crushed with an electric crusher respectively;
then weighing 1.0 g of the crushed sample, and 10 mL of methanol is added, ultrasonic extracting in a water bath at 25° C. for 30 min, taken out and then centrifuged at 5000 r/min for 5 min;
after the centrifugation, taking 5 mL of supernatant of the upper layer and charging it into a syringe, compressing plunger rod of the syringe to enable the supernatant to pass through a 0.45 μm nylon filter membrane, and the resulting sample supernatant could be directly used for HPTLC spotting;
(3) HPTLC spotting and chromatographic conditions:
firstly, manually pipetting the sample solution with a 100 μL spotting needle;
spotting by a semi-automatic thin-layer spotting device, with the assistance of a 0.5 MPa nitrogen flow, sweeping the sample solution to a position 10 cm away from the bottom of the thin-layer plate with a liquid flow sweeping speed of 100 μL/s, a pre-discharge volume of 0.2 μL, a band width of 6 mm and a distance from both side edges of at least 15 mm;
spotting each of the samples prepared by the steps (1) and (2), after finishing the spotting of one sample, manually taking out the spotting needle, washing it with methanol for three times, and then carrying out the spotting of a next sample;
after finishing all the spotting, taking out the thin-layer plate and heated with an electric hair dryer for 1 min to volatilize the residual methanol attached at spotting positions;
Carrying out chromatographic separation in a full-automatic thin layer chromatographic expander with a mobile phase ration: a volume ratio of ethyl acetate/methanol being 9/1 and an expanding distance being 60 mm;
the chromatographic separation conditions:
controlling relative humidity in an expanding tank by bubbling a saturated magnesium chloride solution for 3 min, so as to adjust the relative humidity to 35%, and pre-equilibrating the thin-layer plate for 10 min; when a front edge of the mobile phase reaching a predetermined height, the system automatically ending, taking out the thin-layer plate and baking it on a thin-layer heater at 80° C. for 5 min to obtain a bright and low-noise bioluminescence imaging background;
(4) detection by bioluminescence imaging: immersing the expanded and dried thin-layer plate into a luminescent working suspension by using an automatic immersion device at an immersion speed of 1 mm/s and a retention time of 2 s, then putting the thin-layer plate immersed with the luminescent working suspension into a bioluminescence imager to detect imaging, with a imaging exposure time of 40 s, a imaging interval of 2 min, and taking 15 photographs continuously; and
(5) analysis: saving the photographs taken by the bioluminescence imager, opening the photographs with a Videoscan software, digitizing the pixel gray scale in the photographs, and then setting integral parameters and conditions for quantitative analysis.

3. The method for screening adulteration of fibrate anti-hyperlipidemia chemicals in tea by combined method of high performance thin layer chromatography and bioluminescence according to claim 2, wherein the preparation process of the luminescent working suspension is as follows:
(1) formulation of simulated seawater liquid medium:
formulating a simulated seawater liquid medium according to the following formula: 30 g/L of NaCl, 5 g/L of Na₂HPO₄, 5 g/L of KH₂PO₄, 3 mL/L of glycerol, 5 g/L of peptone and 5 g/L of a yeast extract, adding 1 L of ultrapure water to dissolve under stirring; adjusting the pH value to 7.3-7.7 with 1 mol/L of sodium hydroxide solution, and
carrying out sterilization treatment at 121° C. for 15 min within a high-pressure steam sterilization pot to obtain a simulated seawater liquid medium, then packaging the simulated seawater liquid medium and refrigerating it in a refrigerator for later use, and the simulated seawater liquid medium could be stored in an environment of 4° C. for 7 days when being idle; and
(2) culture and preserve of luminous strains:
inoculating luminous bacteria cryopreserved with glycerol into a triangular flask containing 100 mL of the liquid medium prepared in the step (1);
wrapping the flask mouth with sterilized four-layer folded tin foil paper to ensure external oxygen could enter the flask during the culture process, and then culturing the bacteria in the flask under shaking at 100 r/min in an environment of 25° C. to obtain a bacterial mother liquor; then adding an equal volume of fresh liquid medium into the mature bacterial mother liquor to prepare a luminescent working suspension; and
the luminescent working suspension can be stored in an environment of 4° C. for 3 days when being idle.

4. The method for screening adulteration of fibrate anti-hyperlipidemia chemicals in tea by combined method of high performance thin layer chromatography and bioluminescence according to claim 3, wherein the luminous strains of the step (2) are preserved by an agar plate method, specifically as follows:
a. preparation of luminous bacteria medium:
taking 10 g of agar, 30 g of NaCl, 5 g of Na₂HPO₄, 5 g of KH₂PO₄, 3 mL of glycerol, 5 g of peptone and 5 g of a yeast extract, and adding 1 L of ultrapure water to dissolve under stirring, then adjusting the pH value to 7.5±0.2 with a 1 mol/L sodium hydroxide aqueous solution, and then
carrying out sterilization treatment at 121° C. for 15 min within a high-pressure steam sterilization pot; and
when a sterilized culture medium cooled to 60° C., pouring it into a Petri dish with a diameter of 10 cm to form a plate while hot; and
b. inoculation: firstly, immersing a sterilized inoculating loop into a mature luminous bacteria medium, and then shading with diagonal lines on the surface of the nutrient agar medium, repeating it for many times; culturing the inoculated nutrient agar medium in an environment of 25° C. in dark for 48 h, a bacterial mother liquor is obtained after obvious colonies being observed, and then transferring it into an environment of −4° C. in dark for preservation.

5. The method for screening adulteration of fibrate anti-hyperlipidemia chemicals in tea by combined method of high performance thin layer chromatography and bioluminescence according to claim 3, wherein in the step (2), the absorbance of the liquid medium to an incident light at 600 nm is taken as an index for determining a cell density; a clear simulated seawater liquid medium is taken as a reference, the absorbance OD₆₀₀ of a medium to the incident light at 600 nm is monitored by a spectrophotometer during the culture process, and the medium with the value of OD₆₀₀ reaching 0.7 is selected as the bacterial mother liquor.

6. The method for screening adulteration of fibrate anti-hyperlipidemia chemicals in tea by combined method of high performance thin layer chromatography and bioluminescence according to claim 2, wherein the thin-layer plate in the step (3) needs to be pre-washed, specifically as follows: firstly, pouring 10 mL of methanol into a clean expanding tank, then a blank thin-layer plate being put; expanding the thin-layer plate to the top end thereof, keeping for 5 min when the thin-layer plate expanded to the top end to wash off impurities as many as possible; then taking out the thin-layer plate and baking it on a thin-layer plate heater at 100° C. for 5 min to volatilize the residual organic solvent; and wrapping the dried thin-layer plate with aluminum foil paper for later use.

7. The method for screening adulteration of fibrate anti-hyperlipidemia chemicals in tea by combined method of high performance thin layer chromatography and bioluminescence according to claim 2, wherein the thin-layer material of the thin-layer plate is an ordinary silica gel.

8. The method for screening adulteration of fibrate anti-hyperlipidemia chemicals in tea by combined method of high performance thin layer chromatography and bioluminescence according to claim 2, wherein the specific process of the step (5) is as follows: saving the photographs taken by the bioluminescence imager in CPF, Black/white linear formats, opening the photographs with a Videoscan software, digitizing the pixel gray scale in the photographs, and then setting integral parameters and conditions for quantitative analysis.

9. The method for screening adulteration of fibrate anti-hyperlipidemia chemicals in tea by combined method of high performance thin layer chromatography and bioluminescence according to claim 1, wherein the luminous bacteria, a class of microorganisms, could be emitting visible light under normal physiological conditions.
